(19) United States
(12) Patent Application Publication
(10) Pub. No.: US 2004/0224326 A1
(43) Pub. Date: Nov. 11, 2004

(54) Method of replicating nucleic acid array

(76) Inventors: Young-A Kim, Gyeonggi-do (KR); Hee-Kyun Lim, Gyeonggi-do (KR); Young-Hwan Lee, Gyeonggi-do (KR); Geun-Bae Lim, Gyeonggi-do (KR)

Correspondence Address: CANTOR COLBURN, LLP, 55 GRIFFIN ROAD SOUTH, BLOOMFIELD, CT, 06002

(21) Appl. No.: 10/749,419

(22) Filed: Dec. 31, 2003

(30) Foreign Application Priority Data
Jan. 3, 2003 (KR) 2003-381

(51) Int. Cl. C12Q 1/68; C12P 19/34

(52) U.S. Cl. 435/6; 435/91.2

(57) ABSTRACT

Provided is a method of replicating a nucleic acid array, the method including: (a) manufacturing a template nucleic acid array by immobilizing on a surface of a first substrate first nucleic acid probes, each of which includes a first polynuclotide that has a sequence complementary to a second polynuclotide to be synthesized and a primer binding site; (b) binding a primer to the primer binding site of each of the first nucleic acid probes immobilized on the surface of the first substrate of the template nucleic acid array; (c) in-situ synthesizing a second polynucleotide initiating from the primer using the first polynucleotide as a template; and (d) transferring second nucleic acid probes, each of which includes the second polynucleotide and the primer, to a second substrate from the first substrate.

### BACKGROUND OF THE INVENTION

This application claims the priority of Korean Patent Application No. 2003-381, filed on Jan. 3, 2003, in the Korean Intellectual Property Office, the disclosure of which is incorporated herein in its entirety by reference.

1. Field of the Invention

The present invention relates to a method of replicating a nucleic acid array, and more particularly, to a simple, non-photolithographic, non-spotting method of replicating a nucleic acid array.

2. Description of the Related Art

Nucleic acid arrays are chip-like hybrid devices manufactured by binding biological materials, such as DNA or RNA, to an inorganic material, such as a semiconductor.

Nucleic acid arrays are receiving great attention due to an advantage of offering massive nucleic acid information over conventional sequencing methods. One of important considerations in the manufacture of nucleic acid arrays is to increase the density of integration of biomolecules, for example, nucleic acids, in a limited micrometer-scale area for greater nucleic acid decoding capacity. Currently, nucleic acid arrays with a million of probes are available.

Methods of manufacturing nucleic acid arrays are roughly classified into in-situ methods of directly synthesizing oligonucleotides or cDNA on a chip substrate and methods of spotting previously synthesized nucleotides or cDNA on a chip substrate.

In in-situ methods in which probes are synthesized through direct direction on a substrate by photolithography, it is impossible to correct wrong DNA or RNA probes. In addition, four masks are required to layer four kinds of bases, adenine, guanine, cytosine, and thymine, and repeated buffer exchanging and washing processes are required. Thus, the time and costs required for manufacturing nucleic acid arrays increase in proportion to the length of probes formed.

In spotting methods in which previously synthesized DNAs or RNAs are immobilized after purification, the synthesis of undesired DNA or RNA is prevented. However, sequential spotting of different kinds of nucleic acids is required to manufacture high-density nucleic acid arrays. Therefore, the time required for manufacturing nucleic acid arrays linearly increases with increasing number of kinds of probes to be spotted, thereby making it difficult to mass produce nucleic acid arrays.

### SUMMARY OF THE INVENTION

The present invention provides a method of replicating a nucleic acid array that ensures cost- and time-effective mass production independent of the variety and length of probes to be immobilized.

In one aspect of the present invention, there is provided a method of replicating a nucleic acid array, the method comprising: (a) manufacturing a template nucleic acid array by immobilizing on a surface of a first substrate first nucleic acid probes, each of which includes a first polynuclotide that has a sequence complementary to a second polynuclotide to be synthesized and a primer binding site; (b) binding a primer to the primer binding site of each of the first nucleic acid probes immobilized on the surface of the first substrate of the template nucleic acid array; (c) in-situ synthesizing a second polynucleotide initiating from the primer using the first polynucleotide as a template; and (d) transferring second nucleic acid probes, each of which includes the second polynucleotide and the primer, to a second substrate from the first substrate.

According to specific embodiments of the present invention, the manufacturing of the nucleic acid array may include patterning or treating a surface of the first substrate prior to the immobilization of the first nucleic acid probes. The first substrate may have a metallic pattern made of gold, platinum, or silver. The surface of the first substrate may be treated such that a functional group or a material that can bind to a terminal of the first nucleic acid probes is attached to the surface of the first substrate. Non-limiting examples of the functional group and the material that can specifically bind to a terminal of first nucleic acid probes include aldehyde, streptavidin, thiol, etc.

The template nucleic acid array may be manufactured by contacting protruding portions of the first substrate that is patterned with a first nucleic acid probe solution that fills recessed portions of another substrate with an uneven pattern.

The primer in step (b) may be a universal primer. The primer may have a sequence that does not overlap the second polynucleotide.

A functional group or a material that can bind to a surface of the second substrate may be attached to a terminal of the primer. The method may further include cleaving hydrogen bonds between the first and second polynucleotides before step (d). The hydrogen bonds may be cleaved using a known method, for example, heating above a temperature Tₘ, raising pH, or adding an organic compound such as formaldehyde.

The method according to the present invention may further include patterning or treating a surface of the second substrate prior to transferring the second polynucleotide to the second substrate. The same patterning and surface treatment methods as applied to the first substrate may be used.

Steps (b) through (d) may be repeated using the template nucleic acid array to produce a number of nucleic acid arrays.

### BRIEF DESCRIPTION OF THE DRAWINGS

The above and other features and advantages of the present invention will become more apparent by describing in detail exemplary embodiments thereof with reference to the attached drawings in which:

FIG. 1A illustrates the step of manufacturing a template nucleic acid array;

FIG. 1B illustrates the step of binding a primer to a primer binding site of a first nucleic acid probe of the template nucleic acid array immobilized on a first substrate;

FIG. 1C illustrates the step of in-situ synthesizing a second polynuclotide initiating from the primer;

FIG. 1D illustrates the step of manufacturing a complete nucleic acid array by transferring a second nucleic acid probe, which consists of the second polynucleotide and the primer, to a second substrate;

FIG. 2 illustrates the immobilization of first nucleic acid probes by contacting protruding portions of the patterned first substrate with a first nucleic acid probe solution that fills recessed portions of another patterned substrate;

FIG. 3 is a scanned image of a glass substrate after the in-situ synthesis of a second polynuclotide thereon in Example 1, which was obtained using a fluorescent scanner (GSI Lumonics);

FIG. 4 is a scanned image of a glass substrate after the hybridization of the second DNA probes in Example 2, which was obtained using the fluorescent scanner;

FIG. 5 is a scanned image of a glass substrate with a template nucleic acid array after the transfer of the second DNA probes in Example 2, which was obtained using the fluorescent scanner;

FIG. 6 is a scanned image of an aldehyde-coated glass substrate with the second DNA probes transferred thereto before washing in Example 2, which was obtained using the fluorescent scanner; and

FIG. 7 is a scanned image of the aldehyde-coated glass substrate with the second DNA probes transferred thereto after washing in Example 2, which was obtained using the fluorescent scanner.

### DETAILED DESCRIPTION OF THE INVENTION

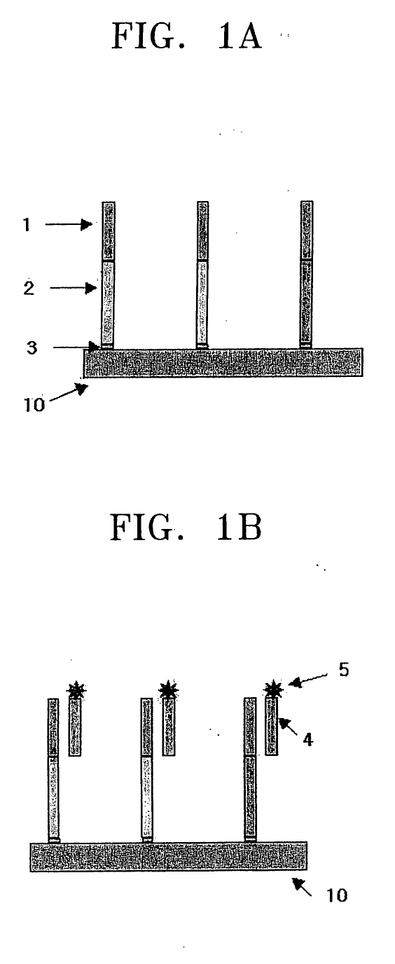

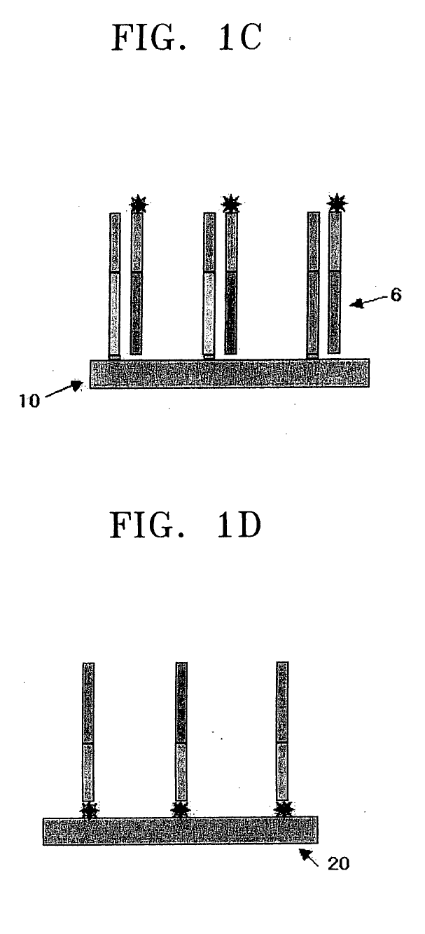

A method of manufacturing a nucleic acid array according to an embodiment of the present invention will now be described step by step with reference to FIGS. 1A through 1D.

Referring to FIG. 1A, which illustrates the step of manufacturing a template nucleic acid array, first nucleic acid probes each of which is comprised of a first nucleotide 2 and a primer binding site 1, the first nucleotide 2 having a sequence that is complementary to a second nucleotide (not shown), is synthesized and immobilized on a surface of a first substrate 10.

The surface of the first substrate 10 may be previously patterned or treated for specific binding with the first nucleic acid probes. When the surface of the first substrate 10 is patterned, a metallic pattern 3 may be formed on the first substrate 10. When the surface of the first substrate is treated, a functional group or material that can bind to a terminal of the first nucleic acid probes may be attached to the surface of the first substrate 10.

The metallic pattern 3 may be formed of gold, platinum, silver, or a combination of the forgoing materials by a general photolithography method. In particular, after a metallic layer (not shown) and a photoresist layer (not shown) are sequentially deposited on the first substrate 10, the photoresist layer is exposed to light through a mask to form a photoresist pattern. The metallic layer is etched using the photoresist pattern as a mask into a desired metallic pattern.

Examples of a functional group or material that can specifically bind to a terminal of the first polynucleotide 2 of each of the first nucleic acid probes includes a thiol group, which is for substrates that are patterned with platinum or gold, an amino group, which is for substrates that are surface-treated with aldehyde groups, biotin, which is for substrates that are surface-treated with streptavidin, and the like.

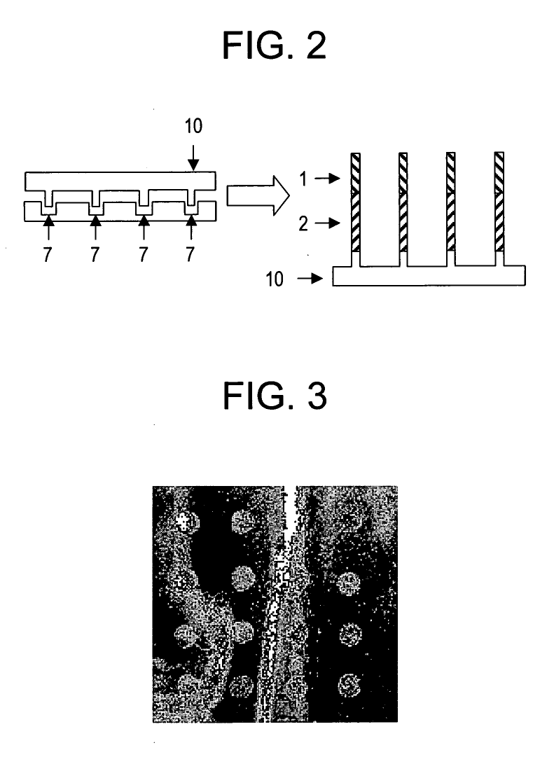

The first nucleic acid probes may be attached to the first substrate 10 as illustrated in FIG. 2. After filling recessed portions of another uneven substrate with a first nucleic acid probe solution, protruding portions of the patterned first substrate 10 are brought into contact with the first nucleic acid probe solution in the recessed portions of the other substrate so that the first nucleic acid probes are attached to a surface of the first substrate 10. When such a patterned first substrate that has protruding portions as probe binding sites is used, many different kinds of nucleic acid probes can be individually and conveniently attached to the first substrate without being mixed together.

The first nucleic acid probes may be uniformly immobilized on a solid substrate as, for example, a self-assembled monolayer (SAM).

FIG. 1B illustrates the step of binding a primer 4 to the primer binding site 1 of the first nucleic acid probe of the template nucleic acid array on the first substrate 10.

The primer 4 may be a universal primer that has a particular base sequence and can bind to nucleic acid primers having various base sequences. The primer binding site of each of the nucleic acid probes immobilized on the solid substrate may have a base sequence that is complementary to the universal primer such that the primer binding site is hybridized with the universal primer.

A number of universal primers that have the same base sequence can be simultaneously hybridized to a number of first nucleic acid probes that have various base sequences at the same temperature. Accordingly, it is easy to control the hybridization temperature. In addition, the degrees of hybridzation of the universal primers to the first nucleic acid probes are almost constant, thereby making it possible to spot uniform lengths of second polynucleotide over a resulting nucleic acid array.

The primer 4 may have a sequence that does not overlap the second polynucleotide for specific binding to the primer binding site 1. A functional group or material may be attached to a terminal of the primer 4 for easy binding to a surface of a second substrate 20 (see FIG. 1D) described later.

FIG. 1C illustrates the step of in-situ synthesizing a second polynuclotide 6 initiating from the primer 4.

A mixed solution of a nucleic acid base elongation enzyme, such as a nucleic acid polymerase, a Taq polymerase, a thermosequenase, etc., the primer 4, and a dNTP is prepared. The thermosequenase is preferred to synthesize a nucleic acid that has a length of 18 mers or less. The Taq polymerase is preferred to synthesize a nucleic acid that has a length longer than 18 mers. As the template nucleic acid array manufactured above is immersed in the mixed solution, the primer 4, which may be a universal primer as described above, hybridizes to the primer binding site 1 of each of the first nucleic acid probes, and simultaneously the second polynucleotide 6 that is complementary to the first polynucleotide 2 is synthesized by extension initiating from the primer 4, thereby resulting in second nucleic acid probes. The second polynucleotides 6 remain bound with the first nucleic acid probes in an appropriate temperature range.

FIG. 1D illustrates a complete nucleic acid array with the second nucleic acid probes, which consists of the synthesized second polynucleotide 6 and the primer 4, transferred to the second substrate 20.

Hydrogen bonds between the first nucleic acid probes of the template nucleic acid array and the second nucleic acid probes are cleaved. The hydrogen bonds may be cleaved by, for example, heating to a temperature Tₘ. The second nucleic acid probes separated from the first nucleic acid probes are transferred and fixed to the second substrate 20.

The second substrate 20 may be patterned or surface-treated for specific binding with the second nucleic acids before the second nucleic acids are transferred to the same. The same methods as applied to pattern and treat the surface of the first substrate 10 may be applied to the second substrate 20. The second substrate 30 to which the second nucleic acid probes, which include the second polynucleotide and the primer, have been transferred is a complete, final nucleic acid array.

A plurality of nucleic acid arrays may be mass produced by repeating the above-described hybridization, in-situ synthesis, and transferring processes with the previously manufactured template nucleic acid array, which is the first substrate with the first nucleic acid probes.

Hereinafter, the present invention will be described in greater detail with reference to the following examples. The following examples are for illustrative purposes and are not intended to limit the scope of the invention.

### EXAMPLE 1

### In-situ Synthesis of Second Polynucleotide on Solid Substrate

A. Immobilization of DNA Probes

DNA probes with amino groups (5′-AGCGTCCTGTTGGTGCTACTACTC TTCTTG-3′-NH₂) were dissolved in a spotting solution (1× Micro-Spotting Solution Plus, available from TeleChem Co.). The solution was spotted over a superaldehyde-coated slide glass (first substrate, available from TeleChem Co.) using a micropipette. The spotted slide glass was left at room temperature (about 25° C.) for 12 hours for drying. To remove non-immobilized DNAs the slide glass was stirred in a 0.2% sodium dodecyl sulfate (SDS) solution for 2 minutes at room temperature twice. The slide glass was rinsed with distilled water twice.

B. In-situ Synthesis of Target DNA

A target DNA was in-situ synthesized using the slide glass manufactured in A and a hybridization solution (DNA solution) at 37° C. for 1 hour. 100 μL of a mixed solution in distilled water of 1.8 μL of a thermosequenase (32 units), 10 μL of a 10× enzyme buffer solution (750 mM Tris-HCl (pH 9.0), 150 mM (NH₄)₂SO₄, 25 mM MgCl₂, 1 mg/mL BSA), 200 μM dNTPs (including Cy3-labeled dUTP), 0.5 mM MgCl₂, and 25 μM of a biotin-labeled universal primer (biotin-5′-caagaagagtagtag-3′) solution was used as a DNA solution for hybridization.

After in-situ synthesis, it was confined whether 15 mers of new DNA was extended from the DNA primer by identifying Cy3-dUTP complementary to a terminal adenine base of the first nucleic acid probe using a fluorescent scanner (GSI Lumonics). A resulting fluorescence scanning photograph is shown in FIG. 3. FIG. 3 illustrates the result of the in-situ synthesis performed using 16 identical DNA probes immobilized on a single chip. 16 fluorescent spots appearing in FIG. 3 support that the second nucleotide probes were successfully synthesized at all of the 16 probe spots in the chip.

C. Transfer of Target DNA to Second Substrate

A 1× Micro-Spotting Solution Plus (TeleChem) solution was applied to the slide glass (first substrate) after hybridization, and another substrate (second substrate) that had been previously coated with streptavidin was laid on a surface of the first substrate where hybridization had occurred. The substrates were left at room temperature for 30 minutes to allow binding of the streptavidin and the biotin and then in a 70° C. oven for 1 hour to cleave hydrogen bonds between the first DNA probes on the first substrate and the second DNA probes on the second substrate. The second substrate with the second DNA probes that were bound to the streptavidin was separated from the first substrate, washed in a 0.2% SDS solution, and dried, so that only the second DNA probes were transferred to the second substrate.

### EXAMPLE 2

### Transfer and Immobilization of Target DNA on Second Substrate

A. Washing of Pt-patterned Glass

A glass substrate with platinum (Pt) patterns (2×2 mm) was washed in a 3:1 mixed solution of H₂SO₄ and H₂O₂ at 90° C. for 10 minutes. The glass substrate was rinsed with triple distilled water and dried using nitrogen.

B. Immobilization of DNA Probes

The Pt-patterned glass substrate was immersed in a 2.5 μM solution of DNA probes (5′-HS-GTTCTTCTCATCATC-3′) in TE buffer (pH 7.4) at room temperature for 3-5 hours to form self-assembled monolayers (SAMs) of the probes. After the reaction, unreacted probes were washed off with triple distilled water.

C. Hybridization of Target DNA

A 2 μM solution of target DNA (3′-NH₂-CAAGAAGAGATAGTAG-FITC-5′), which had a complementary sequence to the DNA probes, a terminal with an amino group, and the other terminal labeled with fluorescein isothiocyanate (FITC), in TE buffer (1M NaCl, pH 7.4) was prepared and subjected to hybridization for 2 hours.

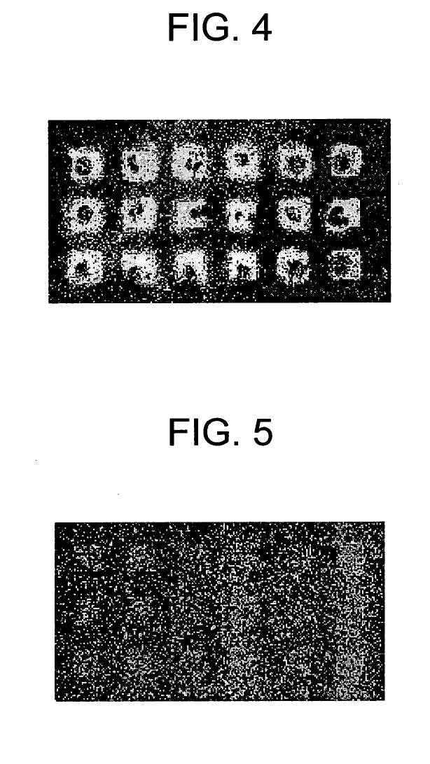

After hybridization, unreacted target DNA was washed off with 2× standard saline-citrate (SSC) containing 2% SDS. Whether hybridization had occurred or not was confirmed using a fluorescent scanner (GSI Lumonics). The results are shown in FIG. 4. Eighteen fluorescent signals from the FITC in FIG. 4 confirm that the DNA probes immobilized on 18 Pt patterns had hybridized with the target DNA.

D. Transfer of Target DNA to Second Substrate

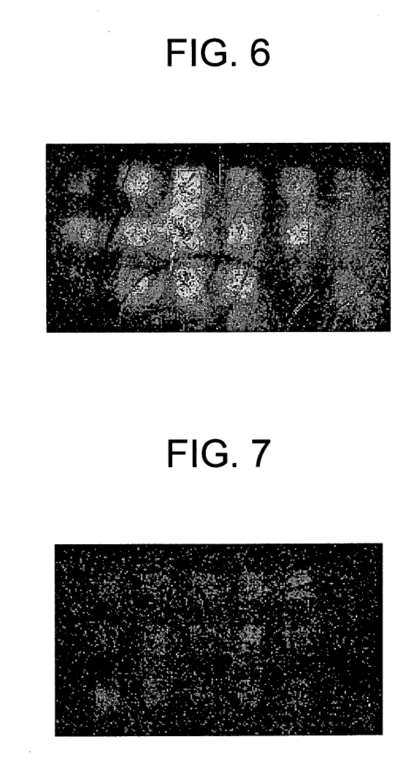

A 1× Micro-Spotting Solution Plus (TeleChem) solution was applied to the Pt-patterned glass substrate after hybridization, and another glass substrate (second substrate) that had been previously coated with aldehyde was laid on a surface of the first substrate where hybridization had occurred. The substrates were left at room temperature for 2 hours to allow CH₂-NH bonding and then in a 80° C. oven for 1 hour to cleave hydrogen bonds between the DNA probes (first DNA probes) on the Pt-patterned glass substrate and the DNA probes (second DNA probes) hybridized to the first DNA probes. The second substrate with the second DNA probes that were bound to the aldehyde was separated from the first substrate, washed in a 0.2% SDS solution, and dried, so that only the second DNA probes were transferred to the second substrate. The Pt-patterned glass substrate after the transfer of the second DNA probes (see FIG. 5) and the aldehyde-coated glass substrate after the transfer of the second DNA probes before and after washing (see FIGS. 6 and 7) were scanned by using a fluorescent scanner (GSI Lumonics). The second DNA probes transferred to the second substrate were quantitated by detecting the FITC previously bound to the target DNA.

According to the present invention, a second polynucleotide is synthesized using one template nucleic acid array and transferred and fixed to another substrate. Therefore, the manufacturing costs and time are reduced over conventional methods independent of the variety and length of probes on a desired nucleic acid array, solving the problems with conventional photolithography and spotting methods.

When universal primers are used, a constant length of second polynucleotide can be synthesized on every nucleic acid probe, resulting in a nucleic acid array with uniform nucleic acid spot size.

Furthermore, according to the present invention, the template nucleic acid can be repeatedly used for easy mass production of complementary nucleic acid arrays.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. A method of replicating a nucleic acid array, the method comprising:
(a) manufacturing a template nucleic acid array by immobilizing on a surface of a first substrate first nucleic acid probes, each of which includes a first polynuclotide that has a sequence complementary to a second polynuclotide to be synthesized and a primer binding site;
(b) binding a primer to the primer binding site of each of the first nucleic acid probes immobilized on the surface of the first substrate of the template nucleic acid array;
(c) in-situ synthesizing a second polynucleotide initiating from the primer using the first polynucleotide as a template; and
(d) transferring second nucleic acid probes, each of which includes the second polynucleotide and the primer, to a second substrate from the first substrate.

2. The method of claim 1, wherein the first and second substrates are previously patterned or surface-treated.

3. The method of claim 2, wherein a metallic pattern is formed as a result of the patterning, and one of a functional group and a material that can bind to a terminal of nucleic acids to be immobilized on the first or second substrate is attached as a result of the surface treatment.

4. The method of claim 3, wherein each of the functional group and the material is independently selected from the group consisting of aldehyde, streptavidin, and thiol.

5. The method of claim 1, wherein the primer is a universal primer.

6. The method of claim 1, further comprising attaching to a terminal of the primer one of a functional group and a material that can bind to a surface of the second substrate.

7. The method of claim 1, further comprising cleaving hydrogen bonds between the first and second polynucleotides before step (d).

8. The method of claim 1, wherein steps (b) through (d) are repeated using the template nucleic acid array to produce a number of nucleic acid arrays.
